(19) United States
(12) Patent Application Publication
(10) Pub. No.: US 2021/0113142 A1
(43) Pub. Date: Apr. 22, 2021

(54) SYSTEM FOR DIAGNOSIS OF TRAUMATIC BRAIN INJURY

(71) Applicants: Michael David Whitt, San Luis Obispo, CA (US); Christopher Marlo Heylman, San Luis Obispo, CA (US); Jared Webster Jones, Anderson, IN (US)

(72) Inventors: Michael David Whitt, San Luis Obispo, CA (US); Christopher Marlo Heylman, San Luis Obispo, CA (US); Jared Webster Jones, Anderson, IN (US)

(21) Appl. No.: 17/138,838

(22) Filed: Dec. 30, 2020

Related U.S. Application Data
(60) Provisional application No. 62/956,331, filed on Jan. 2, 2020.

(51) Int. Cl. A61B 5/00; A61B 5/353

(52) U.S. Cl. CPC A61B 5/4064; A61B 5/6803; A61B 5/002; A61B 2560/0214; A61B 2562/0247; A61B 2562/0219; A61B 5/353

(57) ABSTRACT

A medical diagnostic device (100) in different embodiments uses a headband (204) and a beanie cap (304) configured to be affixed about the head of a subject for conducting measurements for diagnosing whether the subject is at risk of a concussion. A transducer (302) determines magnitude of impact on the head of the subject provides deceleration information associated with magnitude of the force via a pressure waveform. An accelerometer (402) in connectivity with a gyroscope (602) provide directional and angular information to determine probability of risk of a concussion. Bluetooth (404 or WiFi (406) connectivity enable different transmission modes of output signals from device (100) to device capable of performing the calculated metrics and providing a digital putout. Headband (204 and beanie cap (304) can be used under a helmet, cap, or head covering of any type or can be worn without any head covering

### CROSS-REFERENCE TO RELATED APPLICATIONS

This application claims the benefit of provisional patent application Ser. No. 62/956,331, filed 2020 Jan. 2 by the present inventors.

### TECHNICAL FIELD

The present invention relates to techniques for detecting a traumatic brain injury or concussion, and more particularly to systems and apparatus for diagnosis of a concussion.

### BACKGROUND

### Prior Art

The following is a tabulation of some prior art that presently appears relevant:

U.S. patents

| Pat. No. | Kind Code | Issue Date | Patentee |
|---|---|---|---|
| 10,092,237 | B2 | Oct. 9, 2018 | Wong et al. |

U.S. Patent Application Publications

| Publication Nr. | Kind Code | Publ. Date | Applicant |
|---|---|---|---|
| 201720124699 | A1 | May 4, 2017 | Lane |

### Nonpatent Literature Documents

Barth, J. T., Freeman J. R., Broshek, D. K., and Varney, R. N., Journal of Athletic Training, “Acceleration-Deceleration Sport-Related Concussion: The Gravity of It All”(July-September 2001)

### CROSS-REFERENCE TO RELATED APPLICATIONS

This application claims the benefit of provisional patent application Ser. No. 62/956,331, filed 2020 Jan. 2 by the present inventors.

### TECHNICAL FIELD

The present invention relates to techniques for detecting a traumatic brain injury or concussion, and more particularly to systems and apparatus for diagnosis of a concussion.

### BACKGROUND

### Prior Art

The following is a tabulation of some prior art that presently appears relevant:

U.S. patents

| Pat. No. | Kind Code | Issue Date | Patentee |
|---|---|---|---|
| 10,092,237 | B2 | Oct. 9, 2018 | Wong et al. |

U.S. Patent Application Publications

| Publication Nr. | Kind Code | Publ. Date | Applicant |
|---|---|---|---|
| 201720124699 | A1 | May 4, 2017 | Lane |

### Nonpatent Literature Documents

Barth, J. T., Freeman J. R., Broshek, D. K., and Varney, R. N., Journal of Athletic Training, “Acceleration-Deceleration Sport-Related Concussion: The Gravity of It All”(July-September 2001)

### BACKGROUND

### Prior Art

A recent study shows that an estimated 3.8 million concussions occur in the United States every year during competitive or recreational sports or other activities. This study adds that this number, however, is believed to be underreported by as much as 50 percent, because oftentimes, a concussion, mild and severe is ignored or undiagnosed. Sometimes, following a brain injury, a person appears to be normal to untrained observers. They did not sustain any cuts, broken bones, or observable physical deformative in the accident or sporting event. The individual need not be “knocked out” or rendered unconscious in order to have sustained a serious, permanent, and disabling traumatic brain injury. The Center for Disease Control and Prevention reports that even a low speed or low impact blow, bump, jolt to the head, collisions in sports or crash can exert sufficient force or movement on the brain to cause a traumatic brain injury (TBI) or concussion, The severity of the TBI can range from mild, where the subject may experience a transient change in their mental state or briefly lose consciousness, to severe, where the period of unconsciousness is extended and may be accompanied by a memory loss.

Because traumatic brain injuries and consequences can worsen rapidly without treatment, there is a need for objective diagnostic testing to assess the situation quickly. There are injury diagnostic systems that use several tests to identify brain injury after the subject receives an injury or a physical event.

Wong et al. show a diagnostic device that features a framed spectacle or glasses computing device with an accelerometer to perform eye, motor, or visual response tests after an injury or physical event of the user. Use of a framed affixed to the head by sidearms or temples bent down at an angle over the ear may not be secure. The frame can slip down the subject's face if the ears are too small or the skin being too oily. Frames made of plastic or metal construction may become uncomfortable, tempting the user to leave them off Also, the structures can obstruct peripheral vision, leading to the user not correctly seeing one side or the other. Lane shows a concussion screening system that includes an illumination unit, an image sensor array, a processing unit, and a system memory. The Lane device displays stimuli on a screen, processes images, and detects the evaluated person's pupil or pupils after experiencing an injury or physical event. The pupils must pass an image acceptance test, stored, and processed before determining concussion status. The Barth et al. article discusses the relationship of acceleration or deceleration and potential concussions with methods that reduce the probability of sports-related concussions. The Barth et al. article describes the use of physics formulas to analyze recorded, time-stamped, and transmitted applied impact to the helmet to develop prevention strategies after the occurrence of such an effect.

### SUMMARY

The diagnostic device configured as wearable headgear calculates two metrics that affect the brain. The device functions to mathematically evaluate impact acceleration-deceleration force and magnitude of initial impact on the head of a user. Output signals of the calculated metrics are transmitted to remote devices capable of performing the metrics and providing a digital output simultaneously with occurrence of the injury event. The headgear includes headband and beanie cap configurations that can be worn under a helmet or head covering of any type or without any head covering.

### DRAWINGS-FIGURES

In the accompanying drawings, closely related figures have the same number but different alphabetic suffixes.

FIGS. 1A to 1C show in perspective and partial cut-away representation, various aspects of the TBI device in a head-wearable configuration per one embodiment.

FIG. 2 shows a breakaway schematic and sectional representation of the TBI device under one embodiment.

FIGS. 3A and 3B show right-side and left-side elevation views of a wearable headgear configuration according to one embodiment.

FIGS. 4A to 4E show a left-side elevation views of head-wearable configurations, a front elevation view of a head-wearable design, and a left-side and a right-side elevation view of both head-wearable models.

FIG. 5 shows a circuit and schematic representation of the TBl diagnostic device of according to an exemplary embodiment.

FIG. 6 shows a pressure-time waveform graph and a derivative of pressure-time waveform defined by the TBI diagnostic device's two metrics.

FIG. 7 shows a circuit and schematic representation of the diagnostic device including various aspects of data transmission.

FIG. 8 shows a circuit and schematic representation of various aspects of the TBI diagnostic device's integrator metrics.

### DETAILED DESCRIPTION

There are several advantages of the TBI diagnostic device over other diagnostic devices. This diagnostic device is not used in conjunction with a wired helmet or require the performance of subsequent tests such as eye, verbal, and motor response, or visual diagnostic tests. Other advantages of one or more aspects will be apparent from a consideration of the drawings and ensuing description.

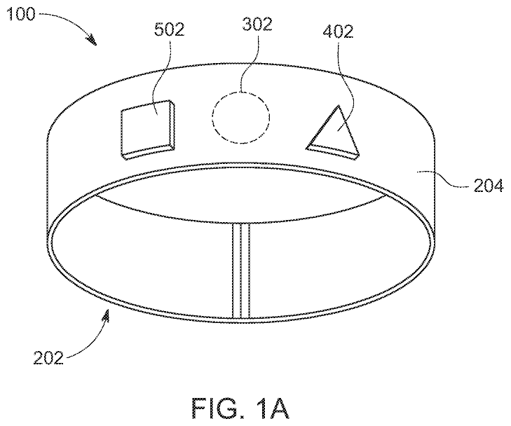

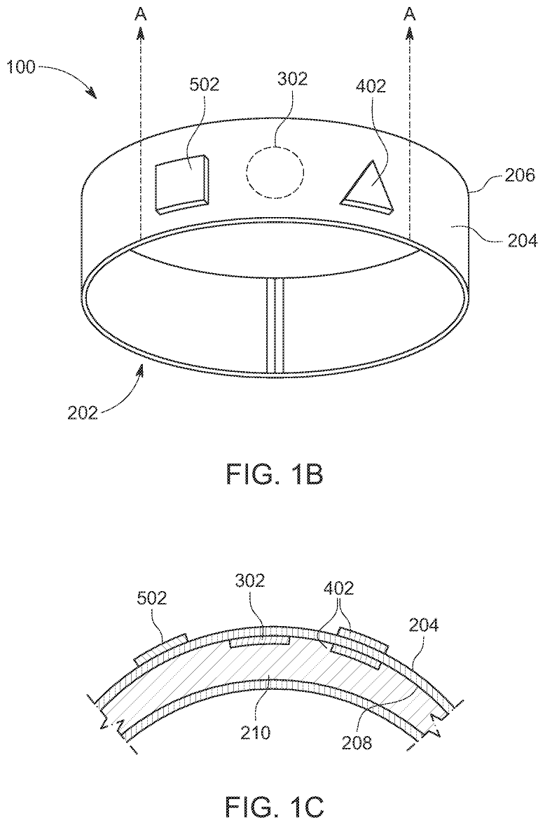

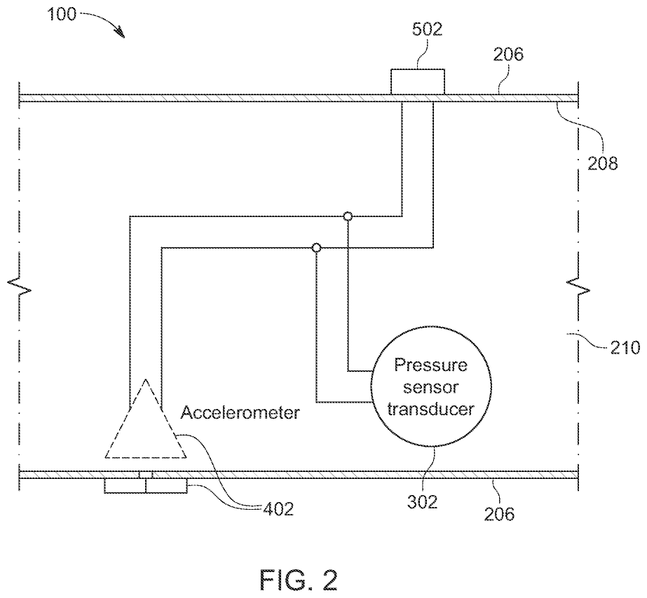

FIGS. 1A to 1C, FIG. 2, FIGS. 3A and FIG. 3B show front perspectives of one embodiment of TBI diagnostic device 100. The device comprises a wearable headgear element 202 configured as a cylindrical headband 204 configured to be wrapped about the head of a subject. Headband 204 is made of any flexible rubber or plastic material. Headband 204 is constructed with an expandable chamber 208 configured to form a substantially fluid-tight seal. Chamber 208 extends the length and width of headband 204 and contains an outside surface 206 closed with at least one membrane. Chamber 208 is filled with incompressible liquid gel substance 210 such that the density does not change with changes in pressure. The liquid gel substance 210 will not leak or loose pressure when compressed or subjected to an external pressure or force.

In an exemplary embodiment, a pressure sensor/transducer 302 operatively connected to an accelerometer/decelerometer 402 are disposed within the interior of headband 204 on chamber 208 enclosure wall. Here, pressure sensor/transducer 302 configured as a Bluetooth® pressure transducer designed to communicate via Bluetooth® transmission mode. A single energy source or power supply 502 is located on headband 204 exterior surface 206 to power both the transducer and accelerometer. The liquid gel substance 210 is maintained in fluid communication with appropriate connectors to Bluetooth® pressure transducer 302 accelerometer 402 and energy source 502 elements.

In another embodiment, pressure transducer 302 connectivity uses a WiFi transmission mode (rather than Bluetooth®) that results in a higher power consumption. When pressure transducer operates in the WiFi transmission mode, pressure sensor 302 component is located inside Chamber 208. Accelerometer 402 can be situated either on the interior or on the exterior of the headband 204.

TBI diagnostic device 100 is operable to conduct pressure, and force impact measurements to the head of the user of the wearable headgear 202. Each of the TBl diagnostic device 100 components, pressure sensor 302, accelerometer 402 and power source 502, serves function for determining the probability of sustaining a concussion for a given force impact. These three elements are arranged and configured to convert input rotational acceleration or deceleration impact pressure into a measurable electronic signal. The electrical signal output is capable of being displayed, and also simultaneously transmitted to a far-end recipient, either by Bluetooth® wireless transmission mode, or by a wired data transmission mode in a manner more fully detailed hereinafter. When the pressure sensor 302 operates in the Bluetooth® transmission mode, the combination of components automatically logs pressure and intensity information. In this arrangement, the Bluetooth® allows transmission of pressure information via the wireless connectivity connection.

Referring now to FIGS. 1A to 1C and FIG. 2, there is disclosed a schematic representation of the device 100 in accordance with an exemplary embodiment of the disclosure. Pressure transducer 302 is disposed an interior wall inside the encapsulated chamber 208. Pressure transducer 302 is configured to sense and measure the pressure of the gel substance 210 that fills the chamber 208. The accelerometer 402 can be located in alternate positions either on the inside of chamber 208 or outside on surface 206. The energy source or power supply 502 is positioned outside chamber 208 on surface 206 to singularly provide a source of energy for the pressure transducer 302 and the accelerometer 402. However, multiple power sources could be used in a given embodiment

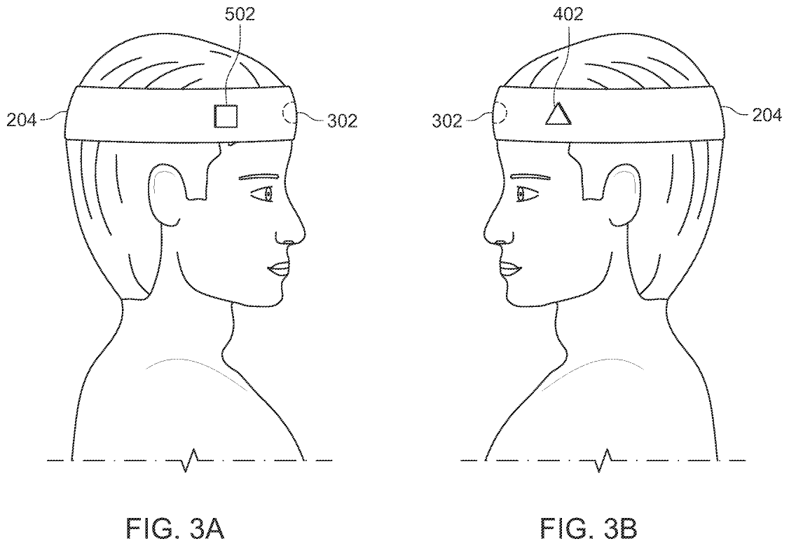

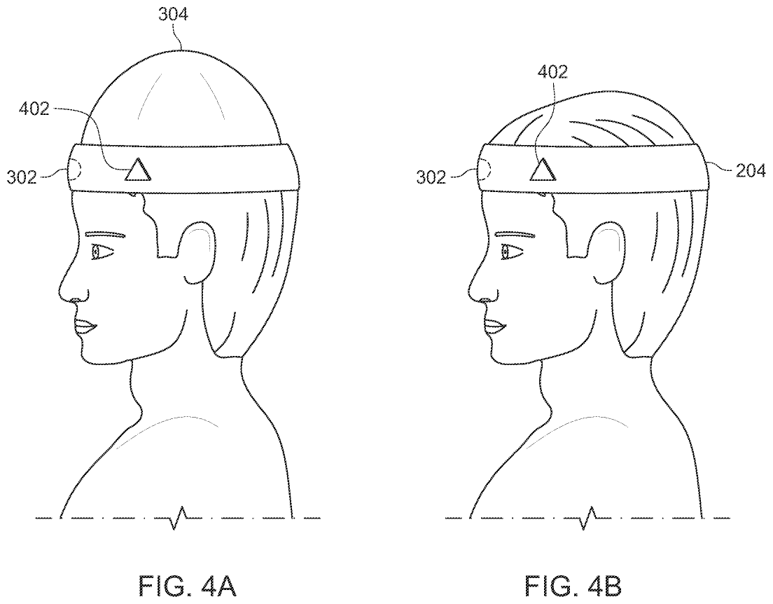

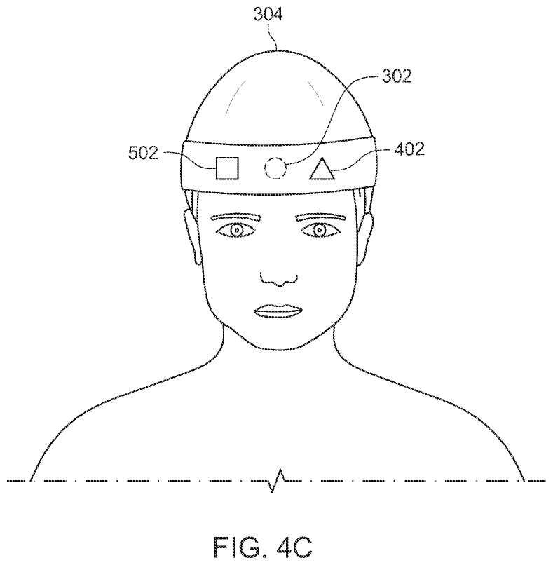

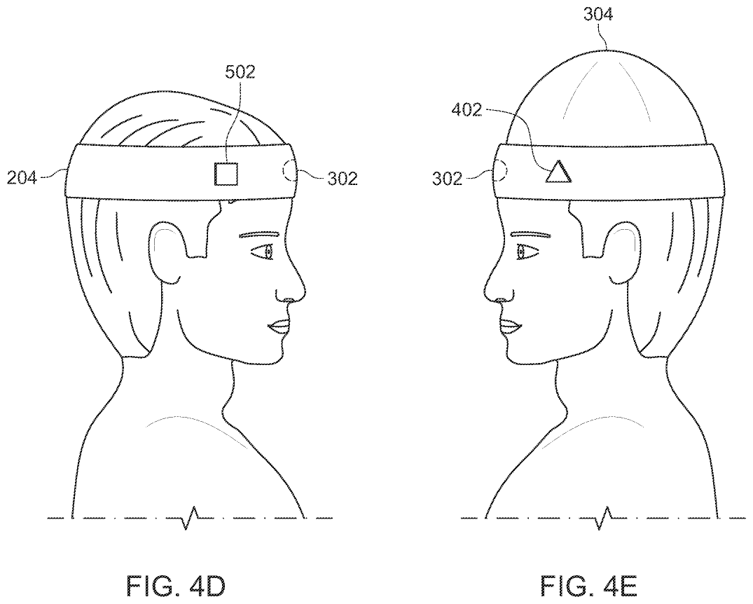

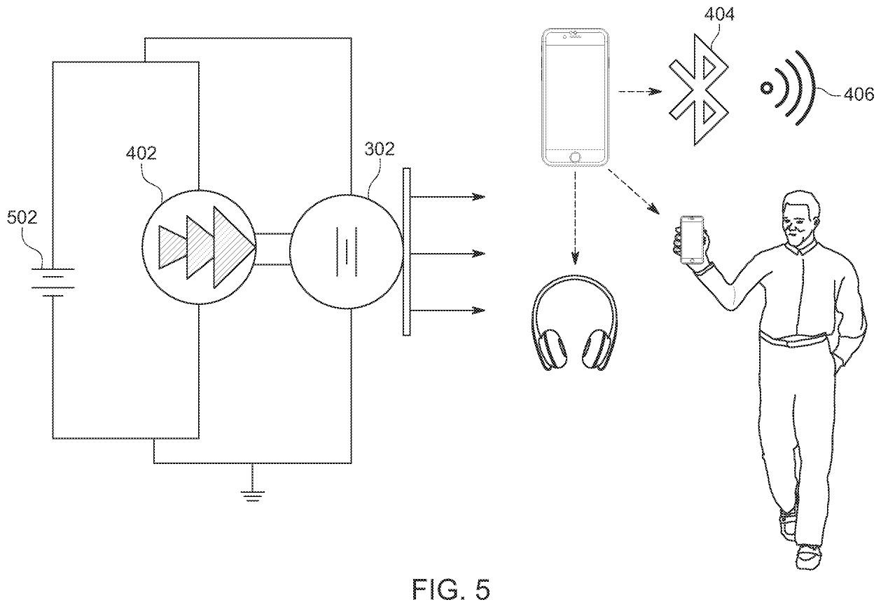

FIGS. 3A and 3B and FIGS. 4A to 4E illustrate the wearable headgear 202 in the form of a cylindrical headband 204 and the form of a beanie or skull cap 304. The cylindrica headband 204 is worn on and affixed around the head of a user. FIG. 3B illustrates a left-side elevation view and a right-side elevation view of the head-wearable element in the form of headband 204 affixed to the head of a user of the device 100. FIG. 4A shows another embodiment where wearable headgear 202 is a skull or the beanie cap 304. Both the headband 204 and the beanie cap 304 are placed on the head of a user in a position to receive a hit, blow or physical impact about the head of the user.

Referring now to FIGS. 1 to 4, a circle is a geometrically representations pressure transducer 302 a triangle represents accelerometer 402 and a square geometric shape represents energy source 502. However, other geometrical shapes are suitable substitutes.

Pressure transducer 302 is physically arranged to always be located inside headband 204 and beanie cap 304 on chamber 208 interior wall. Accelerometer 402 is located on the exterior surface of both headband 204 and beanie cap 304, or on chamber 208 interior wall. Power supply 502 is physically located on the exterior surface of headband 204 and exterior surface of beanie cap 304 headgear and accessible only from outside headband 204 and beanie cap 304.

Technologically, pressure transducer 302 functions as an electronic circuit that replicates, in the form of an electrical signal, an imposed pressure. Thus, in TBI diagnostic device 100 pressure transducer 302 operated to sense and detect an asserted pressure of wearable headgear 202. In both the headband 204 and beanie cap 304 formats, pressure transducer 302 operates to convert an imposed pressure sensed into an analog electric signal whose magnitude depends upon the pressure applied. Because pressure transducer 302 is utilized to convert pressure into an electrical output signal, it is regarded as a pressure sensor transducer.

Referring now to FIGS. 1 to 5, each element located within the interior or on the exterior of the overall device serves a function. Energy source 502 supplies power to both pressure transducer 302 as well as accelerometer 402. However, there could be multiple power sources in a given embodiment.

Both low power accelerometers and low power pressure transducers exist. For example, a Ceramic Micro Electro-Mechanical Systems (MEMS) pressure transducer designed for wireless pressure monitoring could be one class of pressure transducer used. Capacitive ceramic type pressure sensors operate with power consumptions in the range of 0.5 μW while conventional piezoresistive pressure sensors operate with power consumptions in the range of 2.5 mW. Some wireless accelerometers have their own rechargeable batteries while other wireless Bluetooth® accelerometers function with a CR2032 replaceable power cell. Thus, the accelerometer selected for the diagnostic device 100 can vary from a range of accelerometer models.

Accelerometer 402 measure of acceleration or deceleration has been identified as a metric of importance when determining the probability of a concussion. The standard formula for calculating acceleration or deceleration is the formula for acceleration/deceleration or the equation :

a=(v²−v₀²)/2sg

Terms in the acceleration/deceleration equation are as follows:
a is acceleration or deceleration
v₀ is initial directional speed in a given direction
v is final directional speed following deceleration
s is the distance over which the deceleration took place
g is gravitational force (one g force is equivalent to 9.812 m/s²) is a multiplier of acceleration due to gravity or the ‘g force’.

Accelerometer 402 will determine this information for transmission from diagnostic device 100. In one embodiment, accelerometer 402 allows wireless connectivity and output transmission of motion information via Bluetooth® 404 transmission mode. In Bluetooth® 404 transmission mode, accelerometer 402 will automatically log acceleration/deceleration information.

In one embodiment, pressure transducer 302 utilizes Bluetooth® 404 transmission mode to automatically log input pressure and intensity and wireless transmission of pressure information. For operation via Bluetooth® 404 transmission mode, pressure transducer 302 is located on the outer surface of both headband 204 and beanie cap 304. In another embodiment, pressure transducer 302 is located inside headband 204 and beanie cap 304 on chamber 208 interior wall.

Referring to FIGS. 1, 5 and 6 in an exemplary embodiment, accelerometer 402 connectivity is configured to use WiFi 406 mode of transmission. in the WiFi 406 mode of transmission, higher power consumption is required. For use in the WiFi 406 mode of transmission, the accelerometer 402 element is located and affixed on the outer surface of wearable headgear 202. Accelerometer 402 may also be located and affixed on the interior wall of the chamber 208 enclosure when diagnostic device 100 is used in the WiFi 406 mode of transmission.

Pressure transducer 302 functions as a scalar quantity. Thus, a pressure applied to any point within fluid gel substance 210 that fills chamber 208 interior wearable headgear 202 will be equally transmitted to all points of the interior of the wearable headgear 202 with no decremental losses.

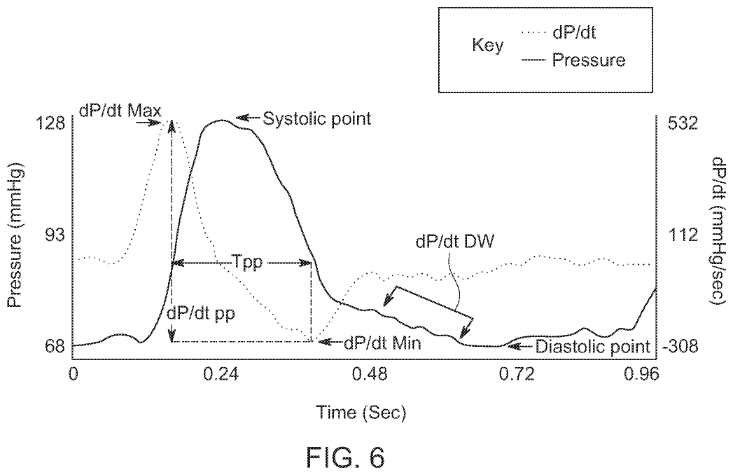

FIG. 6, is a graph of a Pressure-Time Waveform (P) and a Derivative of Pressure-Time Waveform. Two metrics are obtained from the pressure transducer 302. One metric is measured, and the other metric is calculated. The measured metric is graphically depicted as Pressure-Time Waveform (P); and the calculated metric is graphically depicted as a Derivative of Pressure-Time Waveform (dP/dt). FIG. 5 shows an example of what these two waveforms look like from an application where the metrics were used to obtain arterial measurements. The magnitude of pressure waveform will supply information regarding the force associated with the hit to the head while the magnitude of the pressure-time derivative will provide additional information regarding the force and the acceleration/deceleration associated with the force. The algorithm combines all information provided from the accelerometer element and pressure sensor/transducer element towards providing the end-user a probability and/or notification of concussion.

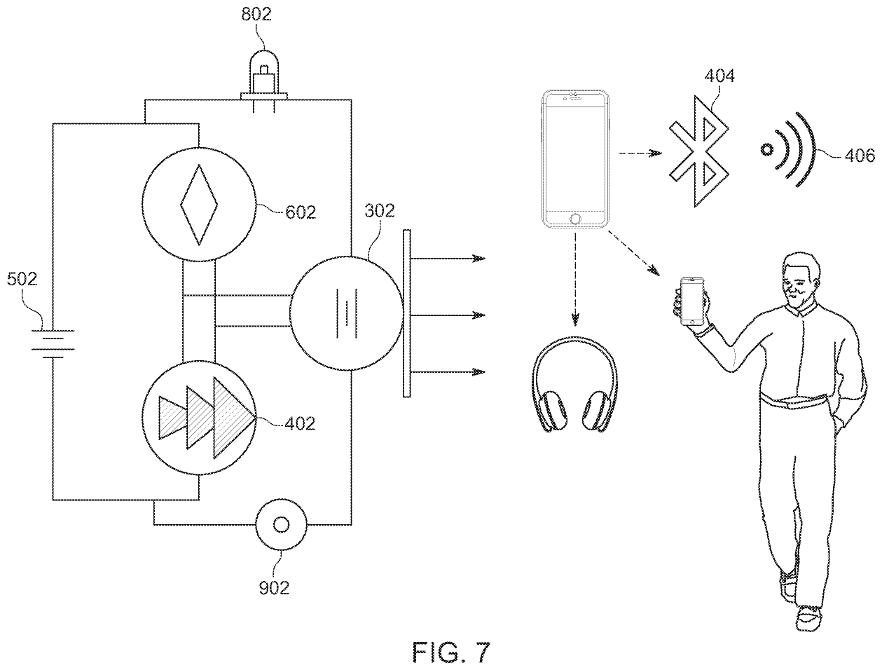

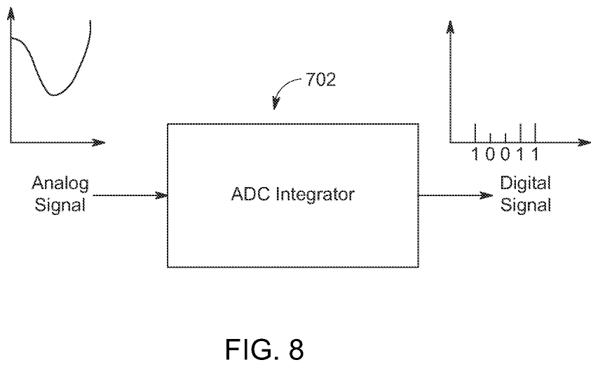

Referring now to FIGS. 5 to 8, a schematic of an exemplary embodiment shows accelerometer 402 connected in tandem with an internal gyroscope 602. In this configuration, gyroscope 602 element is adapted to give additional information useful to provide the end-user with a probability and/or notification of concussion. Gyroscope 602 is operative to transmit angular acceleration/deceleration forces in units converted to radians/sec² relative to the Cartesian Coordinates Reference from accelerometer 402. A LED light source is connected to the device to be triggered to visually alert the end-user and others of the threshold level of a probability of a concussion. Additionally, an audio device 902 is connected to diagnostic device 100 to provide an audible signal to notify the user and others the threshold level of a probability of a concussion. Pressure transducer, accelerometer, and gyroscope signals would be sent to an integrator 702 where the individual signals would be converted from analog to digital (A/D conversion) and amplified if required.

Some possible platforms for integrator 702 include a personal computer, a cellular phone via code embedded within a mobile application, and a computer touch pad via code embedded within a mobile application. Integrator 702 is configured to receive the signal (amplification if needed), convert analog signal to digital signal (A/D Conversion) and display the signal and perform any calculations that result in a calculated metric. Metrics either measured or calculated at integrator 702 include pressure waveform (from pressure transducer); measured metric (amplitude of pressure waveform); and calculated metric (pressure-time derivative maximum value resulting from pressure waveform.

Additional metrics measured or calculated at integrator 702 are linear acceleration/deceleration forces (from accelerometer) of: measured metric gravitational force in x-y-z axes (Cartesian Coordinate System); rotational acceleration/deceleration forces (from gyroscope); and measured metric angular acceleration/deceleration forces (Radians/sec² relative to Cartesian Coordinates Reference from the accelerometer).

The measured and calculated metrics obtained at integrator 702 will be used to generate a function that provides probability of concussion metrics as defined in Table 1 as follows:

| Metric (Measured | Defined | |
|---|---|---|
| or Calculated) | Variable | Description |
| Calculated | Y | Probability of Concussion |
| Measured | X₁ | Pressure Waveform (Pressure |
| | | Waveform Amplitude) |
| Calculated | X₂ | From Pressure Waveform |
| | | (Peak to Peak Magnitude of |
| | | Pressure) |
| Measured | X₃, X₄, X₅ | From Accelerometer: G-Force |
| | | in x-y-z axes (Cartesian |
| | | Coordinate System |
| Measured | X₆ | From Gyroscope: Angular |
| | | Acceleration relative to the |
| | | Cartesian Coordinate |
| | | Reference from the |
| | | accelerometer |

A mathematical model based on the following relationship could be used to indicate probability of concussion is shown in Equation 1.

Y=f(X₁, X₂, X₃, X₄, X₅, X₆)

Equation 1. Framework for Probability of Concussion Mathematical Model Equation

A critical value for “Y” will be identified where the integrator will notify a subject wearing the device headgear as well as those monitoring the device that the subject is at risk for a concussion. This notification could be audible and/or visual. Following notification, the subject would then be prompted to receive proper clinical treatment.

What is claimed is:

1. A device for diagnosing a concussion comprising:
1. a flexible headgear means provided with a chamber inside said flexible headgear means, said chamber extends throughout the length of said flexible headgear means;
2. an incompressible fluid substance that fills said chamber;
3. a pressure sensor transducer disposed on an interior wall within said chamber;
4. an accelerometer in electrical connection with said pressure sensor transducer, said accelerometer positioned on said interior wall within said chamber; and
5. a power source in connectivity with said pressure sensor transducer and said accelerometer, said power source accessible from said flexible headgear mean's exterior.

2. The device of claim 1 wherein said pressure sensor transducer s a Bluetooth® pressure transducer; and wherein said flexible headgear means is made of an elastic material.

3. The device of claim 2 wherein said chamber is sealed from the atmosphere such that said incompressible fluid substance would not leak or lose pressure whenever said flexible headgear means is subjected to an external pressure force.

4. The device of claim 3 further comprising a gyroscope, and wherein said accelerometer is a Bluetooth® accelerometer connected electrically in tandem with said gyroscope to sense, detect and transmit information via a Bluetooth ® mode of transmission.

5. The device of claim 4 wherein said Bluetooth® accelerometer works in tandem with said gyroscope to sense and detect acceleration/deceleration and directionality of an impact to the head of the user of said flexible headgear means; and wherein said Bluetooth® pressure transducer is configured to sense and detect an asserted pressure whose magnitude depends upon the pressure applied on said incompressible fluid gel substance and converts it into an analog electric signal.

6. The device of claim 5 wherein said Bluetooth® accelerometer is configured to mathematically calculate metrics of:
a. asserted acceleration/deceleration of an impact to the head of the user of said flexible headgear means;
b. an initial directional speed;
c. a final directional speed following deceleration;
d. distance over which deceleration took place; and
e. gravitational force (as a multiplier of acceleration/deceleration).

7. The device of claim 6 wherein said Bluetooth® pressure transducer is configured to sense and detect an asserted pressure applied to any point within said incompressible fluid substance contained within said chamber and equally transmitted to all points of said chamber with on decremental loss, and whose magnitude depends upon the pressure applied on said incompressible fluid gel substance.

8. The device of claim 7 wherein said Bluetooth® pressure transducer functions to produce a Pressure-Time Waveform (P) measured metric and a Derivative of Pressure-Time Waveform (dP/dt) calculated metric.

9. The device of claim 8 wherein magnitude of said Pressure-Time Waveform(P) gives information regarding the force associated with a hit to the head of the wearer of said flexible headgear means, and wherein magnitude of said Derivative Pressure-Time Waveform(dP/dt) gives additional information regarding force and acceleration/deceleration associated with force.

10. The device of claim 9 wherein a mathematical model based on the relationship:
Y=f(X₁, X₂, X₃, X₄, X₅, X₆)

11. The device of claim 1 wherein said flexible headgear means is configured in the form of a cylindrical headband and capable of being worn under a helmet if desired.

12. The device of claim 1 wherein said flexible headgear means is configured in the form of a beanie cap and capable of being worn under a helmet if desired.

13. The device of claim 3 wherein said accelerometer connectivity is configured to use a WiFi mode of transmission.

14. A device for diagnosing a concussion comprising:
a wireless transducer to detect a magnitude of a pressure contact via magnitude of a pressure waveform, and an impulse equal to force x time calculation via use of a pressure-time derivative taken from said pressure waveform; a wireless accelerometer-gyroscope operating in tandem to provide directional and angular information; and an energy source cooperating to simultaneously provide output information to a device user and end users whether said device user is at risk of a concussion.

15. The device of claim 14 wherein said user and end users are provided with key information of:
a. magnitude of pressure;
b. magnitude of pressure-time derivative;
c. magnitude of gravitational force deceleration;
d. direction of impact via acceleration data; and
e. angular deceleration via gyroscope.

16. The device of claim 15 wherein different alarm values are calculated for:
a. magnitude of pressure;
b. magnitude of pressure-time derivative;
c. magnitude of gravitational force deceleration;
d. direction of impact via acceleration data; and
e. angular deceleration via gyroscope.

17. A method for diagnosing a brain injury comprising the steps of:
(a) providing a head-wearable device with a cavity or chamber therethrough;
(b) filing the head-wearable device with a liquid gel substance;
(c) attaching an accelerometer device to an exterior surface of the head-wearable device or an interior surface of the chamber;
(d) attaching a pressure sensor device to an interior surface of the chamber;
(e) providing an energy source outside the head-wearable device for maintaining the energy source in contact with the accelerometer and pressure sensor;
(f) affixing the head-wearable device to the head of a user;
(g) measuring the deceleration and magnitude values of an impact to the head of the user; and
(h) performing a mathematical diagnosis whether the combination of deceleration and magnitude values exceeds a threshold value of probability of concussion without performing further procedures.
